(19) United States
(12) Patent Application Publication
(10) Pub. No.: US 2004/0129648 A1
(43) Pub. Date: Jul. 8, 2004

(54) Antimicrobial matrix and method of use

(76) Inventor: Nick J. Manesis, San Diego, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/625,947

(22) Filed: Jul. 23, 2003

Related U.S. Application Data
(60) Provisional application No. 60/397,771, filed on Jul. 23, 2002.

(51) Int. Cl. C02F 1/68

(52) U.S. Cl. 210/764

(57) ABSTRACT

A liquid dispensing apparatus for dispensing a sterile liquid comprises a container for storing the sterile liquid, a dispenser engaged with the container for expressing the sterile liquid from the container and a substrate of a porous, three-dimensional matrix within the container, the substrate providing an anti-microbial coating. The coating provides at least 4 square centimeters of surface area for each cubic centimeter of the interior volume of the container.

### RELATED APPLICATIONS

This application claims priority and is entitled to the filing date of U.S. Provisional application Ser. No. 60/397,771 filed Jul. 23, 2002, and entitled “Antimicrobial Device” The contents of the aforementioned application are incorporated by reference herein.

INCORPORATION BY REFERENCE: Applicant(s) hereby incorporate herein by reference, any and all U.S. patents, U.S. patent applications, and other documents and printed matter cited or referred to in this application.

### BACKGROUND OF THE INVENTION

1. Field of the Invention

This invention relates generally to antimicrobial agents and methods of use and more particularly to an antimicrobial matrix used in dispensing containers for maintaining sterile liquids.

2. Description of Related Art

The following art defines the present state of this field:

Gettings et al., U.S. Pat. No. 5,013,459 describes a method and device for dispensing an aqueous fluid which is desired to be maintained in a sterile condition. The method includes storing a quantity of aqueous fluid such as ophthalmic saline solution in a reservoir within a portable container having an outlet. A porous filter medium is arranged within the container adjacent the outlet, and the aqueous ophthalmic fluid is caused to pass from the reservoir through the porous medium and to the outlet. The porous medium has covalently bonded thereto an antimicrobially effective amount of an organosilicon quaternary ammonium compound which is an organosilane. The organosilane can also be bonded to the inner and outer surfaces of the portable container.

Geimer, U.S. Pat. No. 5,232,687 describes a fluid dispenser incorporating an oligodynamically germicidally active substance for maintaining the dispensation of germ-free fluid, particularly eye drops. The delivery passage from a supply container associated with the fluid dispenser of the invention contains an oligodynamically germicidally active substance that is soluble in the fluid to be dispensed. The device does not require air pressure within the container, thus eliminating one avenue for the introduction of germs into the fluid. The device includes a metering pump and inlet and outlet valves. The above-mentioned fluid soluble oligodynamically germicidally active substance is situated in the region of the inlet valve or the inlet thereto and/or the outlet therefrom.

Sawan et al., U.S. Pat. No. 5,490,938 describes a liquid dispenser for dispensing sterile liquid comprising a container for storing the sterile liquid, a nozzle assembly mounted on the container, and a filter which has at least one surface and a plurality of its pores coated with a metallic material, e.g., a metal or metal oxide or metal salt, that is bacteriostatic or bacteriocidal, is described. The metal coated filter, methods for coating the filter, and methods for using the liquid dispenser are also provided.

Geimer, U.S. Pat. No. 5,614,172 describes a fluid dispenser for germ-free fluid, in particular eyedrops, the delivery passage from a supply container containing an oligodynamically germicidally active substance that is soluble in the fluid. Said device comprises a metering pump operating without air pressure compensation and having an inlet valve for closing the inlet opening from said container and said fluid-soluble oligodynamically germicidally active substance is situated in the region of said inlet valve or of the inlet thereto and/or the outlet therefrom.

Guttag, U.S. Pat. No. 5,648,084 describes a multiple dosage medicine drop bottle or medicinal container having a germicide on at least the outside tip of the bottle or container. The present invention also reveals a method for reducing or eliminating the risk of contamination of the multiple dose medicine drop bottle or medicinal container and it contents. The bottle or container may also contain an aqueous solution of an anticlotting agent.

Sawan et al., U.S. Pat. No. 5,681,468 describes a liquid dispenser for dispensing sterile liquid comprising a container for storing the sterile liquid a nozzle assembly mounted on the container and a filter which has at least one surface and a plurality of its pores coated with a metallic material, e.g. a metal or metal oxide or metal salt, that is bacteriostatic or bacteriocidal, is described. The metal coated filter methods for coating the filter and methods for using the liquid dispenser are also provided.

Sawan et al., U.S. Pat. No. 5,817,325 describes contact killing antimicrobial articles, devices and formulations, which kill microorganisms on contact. The articles, devices or formulations contain a non-leaching antimicrobial material, which is a unique combination of an organic matrix having biocidal metallic materials nonleachably associated with the matrix. The antimicrobial material may be used to form an antimicrobial coating or layer on a surface of the article or device, or may be dispersed in a vehicle or carrier to form a topical antiseptic or disinfectant, or solid shape having contact killing antimicrobial properties. When a microorganism contacts the article, device, or formulation, the biocidal metallic material is transferred to the microorganism in amounts sufficient to kill it.

Sawan et al., U.S. Pat. No. 5,849,311 describes an antimicrobial material, which can be used to form on the surface on a substrate a non-leaching antimicrobial coating or layer which kills microorganisms on contact. The non-leaching antimicrobial coating or layer is a unique combination of an organic matrix immobilized on the surface of the substrate to having biocidal metallic materials non-leachably associated with the matrix. When a microorganism contacts the coating or layer, the biocidal metallic material is transferred to the microorganism in amounts sufficient to kill it.

Sawan et al., U.S. Pat. No. 5,869,073 describes a liquid composition for applying a non-leachable antimicrobial coating on a surface. The liquid composition consists of a solution, dispersion or suspension of a biguanide polymer reacted with a cross-linking agent to form an adduct, and an antimicrobial metal material. The resulting antimicrobial coating does not release biocidal levels of leachables into surrounding solution.

Sawan et al., U.S. Pat. No. 6,030,632 describes an antimicrobial material comprising an antimicrobial metallic material and a biguanide polymer reacted with a crosslinker to form an adduct is described. Both freestanding antimicrobial materials and antimicrobial films are provided. The antimicrobial material does not release biocidal levels of leachables into a contacting solution.

Sawan et al., U.S. Pat. No. 6,126,931 describes contact killing antimicrobial articles, devices and formulations are described which kill microorganisms on contact. The articles, devices or formulations contain a non-leaching antimicrobial material which is a unique combination of an organic matrix having biocidal metallic materials nonleachably associated with the matrix. The antimicrobial material may used to form an antimicrobial coating or layer on a surface of the article or device, or may be dispersed in a vehicle or carrier to form a topical antiseptic or disinfectant, or solid shape having contact killing antimicrobial properties. When a microorganism contacts the article, device, or formulation, the biocidal metallic material is transferred to the microorganism in amounts sufficient to kill it. Sawan et al., U.S. Pat. No. 6,180,584 describes a composition that, when applied to a substrate, forms an adherent, transparent, water insoluble polymeric film on the substrate surface that provides sustained antimicrobial disinfecting action for prolonged periods, without the necessity for reapplication. The coating provides surface disinfecting action by a contact killing mechanism, and does not release its components into contacting solutions at levels that would result in solution disinfection. The polymeric film formed by the composition of the invention can be removed by treatment with dilute alcoholic base.

Schilthuizen et al., U.S. Pat. No. 6,341,718 describes a squeeze bottle for dispensing a liquid, in particular a liquid medicament, in a metered and substantially germ-free manner, at least comprising: a storage chamber for accommodating the liquid, a metering chamber with a variable volume for dispending from this chamber, under reduction of volume of the chamber, a measured volume of liquid, restoring means for returning the metering chamber to its original state after a volume of liquid has been dispensed, an inlet for placing the metering chamber in communication with the storage chamber by mediation of a flow restrictor, which flow restrictor counteracts flow from the metering chamber to the storage chamber substantially completely when the pressure in the storage chamber is increased, counteracts flow between the storage chamber and the metering chamber substantially completely and, when the increase pressure in the storage chamber is removed, allows flow from the storage chamber into the metering chamber, at least one outflow opening for placing the interior of the metering chamber in communication with the environment, the outflow opening comprising a non-return valve, actuating means can be actuated by squeezing the bottle, means for ventilating the squeeze bottle.

Lawson, U.S. Pat. No. 6,579,539 describes compositions, which reduce the possibility of inducing microbial resistance. The compositions comprise a fast-acting antimicrobial agent and a long-lasting antimicrobial agent. The combined effect of the antimicrobial agents reduces microbial infection and resistance.

Eye Research Institute of Retina, WO90/05110 describes a multi-dose dispenser for sterile liquids, which provides antimicrobial action without need for preservatives or antibacterial additives. The conventional manner of preventing microbial growth is to add a preservative or antibacterial agent to the solution. The preservatives themselves are often toxic not just to bacteria but also to the cells, which are being treated by the bottled preparation. A squeeze bottle has a reservoir chamber connected by flow path to tip. A filter assembly is located across flow path near tip. Filter assembly contains a hydrophilic filter near tip, and a hydrophilic filter closer to the reservoir chamber. Both filters have pores, which are of a size sufficient to prevent bacteria from transversing the filter

Kodak Limited, EP0733303 describes a biocidal material comprises an organic biocide immobilized on a polymeric support characterized in that the support is water-insoluble and the biocide is covalently bound to the support by a hydrolytically stable covalent linkage. The material can be used for inhibiting bacterial growth in an aqueous medium e.g. the wash water of a photo processing system. The material can be housed in a flow-through container.

Hsiao, EP1025756 describes a porous formed article capable of killing or inhibiting the harmful microbes, on which carries the antimicrobial component having function to retard the harmful microbes. The said component is selected from the combination comprising the Low Temperature Far Infrared Material and/or the Quaternary Ammonium Salt. In the invention, the porous formed article capable of killing or inhibiting the harmful microbes is resulted from the tight bonding and/or sticking together between the antimicrobial component and the porous formed article having high surface area; therefrom, even after application for long period of time, the active component on it would not be flown away and thus is able to maintain the long effectiveness of the antimicrobial.

Gueret, EP1136056 describes a container having a sealed supple pouch holding a product to be applied with the aid of a porous applicator impregnated with at least one biocide agent which is solid or concentrated when the applicator is dry. The agent can be bactericidal, bacteriostatic and/or anti-fungal, hydrophilic, hydro soluble, liposphilic or liposoluble, and can comprise e.g. salicylic acid, sodium sulphite or methosulfate, benzoic acid, sodium benzoate or other substances.

Our prior art search with abstracts described above teaches antimicrobial agents used in containers for maintenance of sterile conditions but does not teach the use of fine particles or wool-like material in a pocket or pouch to form a matrix, with the matrix coated with a microbial agent, the agent immersed in a sterile liquid. The present invention fulfills these needs and provides further related advantages as described in the following summary.

### SUMMARY OF THE INVENTION

The present invention teaches certain benefits in construction and use which give rise to the objectives described below.

The present invention is a liquid dispensing apparatus for dispensing a sterile liquid comprises a container for storing the sterile liquid, a dispenser engaged with the container for expressing the sterile liquid from the container and a substrate of a porous, three-dimensional matrix within the container, the substrate providing an anti-microbial coating. The coating provides at least 4 square centimeters of surface area for each cubic centimeter of the interior volume of the container or of the liquid volume in the container.

A primary objective of the present invention is to provide an apparatus and method of use of such apparatus that provides advantages not taught by the prior art.

Another objective is to provide such an invention capable of maintaining sterility of a liquid in a container.

A further objective is to provide such an invention capable of inexpensive and easy manufacture.

A still further objective is to provide such an invention capable of complete coating of a substrate followed by compaction to produce an effective matrix.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is an elevational view of a capped dispensing bottle of the invention showing one wall cut away to disclose a sterile liquid and a coated substrate material formed into a matrix;

FIG. 2 is an elevational view of a dispensing bottle with ophthalmic nozzle of the invention showing one wall cut away to disclose the sterile liquid and the coated substrate;

FIG. 3 is an elevational view of a syringe and needle of the invention showing one wall cut away to disclose the sterile liquid and the coated substrate;

FIG. 4 is an elevational view of a closed pocket of fine pore size enclosing small particles coated with an antimicrobial substance and agglomerated to form a matrix;

FIG. 5 is an elevational view of a wool-like material coated with the antimicrobial substance and agglomerated to form a matrix; and

FIG. 6 is an elevational view of a very fine porosity filter paper formed as a wound roll and shown partly unrolled to display details of the sheet from which the roll is formed, the sheet being coated with the antimicrobial substance to form a matrix.

### DETAILED DESCRIPTION OF THE INVENTION

The above described drawing figures illustrate the invention in at least one of its preferred embodiments, which is further defined in detail in the following description.

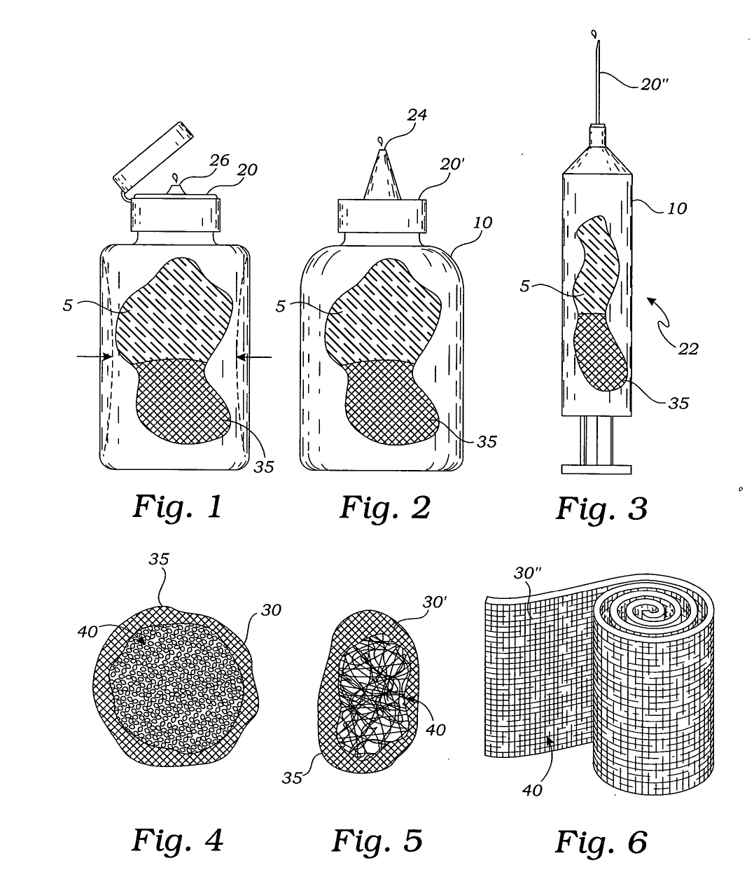

The present invention is a liquid dispensing apparatus for dispensing a sterile aqueous liquid 5 that may contain a medicine or other ingredients and that must remain sterile. Since dispensing containers may not always be able to hold such liquids in the sterile state, there is the need for a container that will provide an antimicrobial environment. The apparatus includes a dispensing container 10 for storing the sterile liquid 5, the dispensing container 10 providing a known useable interior volume. A dispensing means 20 or 20′ or 20″ is engaged with the container 10 for expressing the sterile liquid 5 from the container 10 upon demand or need. Such expression of the sterile liquid 5 may be accomplished by pressing the sides of the container 10, assuming it is made of compliant material as is well known in such containers and in wide use. See the arrows in FIG. 1. This forces the liquid 5 out of the container 10 by pressure. Alternatives include gravity feed and shaking the container 10. The dispensing means 20 may be a needle and syringe 22 as shown in FIG. 3, an ophthalmic tip 24, as shown in FIG. 2 or a nozzle 26 as shown in FIG. 1. Many other forms of dispensing may also be used and are well known in the art.

A substrate 30 or 30′ or 30″ comprising a porous, three-dimensional matrix is contained within the container 10 along with the liquid 5, the substrate providing an anti-microbial coating 40. The coating 40 provides at least 4 square centimeters of surface area exposed to the liquid 5, for each cubic centimeter of the usable interior volume of the container 10.

Preferably, the substrate, referred to here as a matrix is at least one of: small spheres or particles, as shown in FIG. 4, a wool-like material as for instance, glass-wool, shown in FIG. 5, or a compressed high porosity sheet stock made from filter paper, as shown in FIG. 6. The small particles may be made of glass, polymer, silica, diatoms, talc and other materials.

The matrix may also be any one of the well known open-cell structure plastics, such as polyvinyl alcohol and polyurethane or sponges and similar materials. The coating on the matrix 30 may be of any material that provides an antimicrobial action and examples are members of chemical families such as: an alcohol, an anilide, a metal salt, a phenol, a quarternary ammonium compound, a biguanide, an iso-thiazolone, a phenol, a surface-active agent, and, of course, combinations of these substances, or other substances of known antimicrobial value.

By way of definition, the word “matrix” as used here defines any structure that is wholly integral or, alternatively, is made up of tightly packed individual parts or particles. For instance, the matrix of FIG. 4 is formed of many small particles that are packed into a closed pocket 35 so that the particles are in intimate contact one with the other. This matrix then, provides the substrate surface which is the composite large surface of all of the particles taken together, and includes all of the spaces between the particles. The liquid 5 is able to flow easily through the matrix and through the material of the pocket 5, as well, so as to come into contact with the coating on the substrate. This contact destroys any microbes that may exist in the liquid 5. FIG. 5 defines a wool-like material that may be made of a metal, plastic, a glass or other substances, and is coated with the coating 40. Such a wool-like material is also advantageously contained within a pocket 35 so as to achieve a high degree of compaction. It has the same properties as the matrix of FIG. 4, i.e., a large surface area for contact with the liquid and an open structure for liquid flow within and through the matrix. Likewise, in FIG. 6 is shown a loosely wound roll of filter paper or the like, substrate 30″. This roll would be tightly wound for use in the present invention and the sheet stock from which it is made is coated with the antimicrobial coating 40 as defined above. Again, this matrix provides a large surface area and a significant amount of space for liquid to move through the matrix for contacting the coating 40. The creation of a matrix that is immersed in a sterile liquid, the matrix made up of an integral or a composite substrate having a large surface area to volume and with an antimicrobial coating on its entire surface is highly novel in the technology. For instance, Gettings et al, Geimer, Guttag, Rossi and Fueret (FR) all teach the use of a filter or similar device that comes into contact with the liquid only upon dispensing. Hsiao teaches a honeycomb structure of high surface area coated with an antimicrobial and immersed in a sterile liquid. Batts (EU) teaches a polymeric support comprising particles having a biocide covalently bonded to it and restrained from the outflow by a membrane. Sawan et al teaches a non-leaching, antimicrobial layer or coating on a surface such as fibers immersed within a sterile solution. However, the prior art does not teach a composite particulate or wool held integrally within a porous closed pocket, nor a tightly wound filter paper, both of which offer the advantage of being able to be spread-out or unrolled to allow inexpensive and easy coating, while then being able to be gathered together tightly into a matrix form.

The coating 40 is applied by any technique that will provide a permanent tenacious coating, and examples of such methods include the well known: dip coating, spray coating, electroplating, plasma deposition, plasma spraying, and vacuum deposition, or any other method for depositing the coating in a manner where the coating is not likely to degrade or slough-off. Clearly the coating 40 may be held on or adhered to substrate by adsorption, absorption, diffusion, mechanical bonding, chemical bonding, co-polymerization, blending or any other means appropriate to the objectives of the present invention.

In the present invention, a preferred method for dispensing the sterile liquid 5 includes providing the container 10 for storing the sterile liquid 5, the container providing the usable interior volume, and engaging the dispensing means 20 with the container 10, incorporating the substrate 30 within the container, and coating the substrate with the anti-microbial coating 40 to provide at least 4 square centimeters of surface area for each cubic centimeter of the usable interior volume of the container 10. Such a formula is considered to be highly novel in that it has been shown to be effective for all of the materials described in this application and is most probably effective for any other antimicrobial materials as well that have even a small effect on the liquid since the matrix is immersed within the liquid. The method further comprises the step of forming the matrix using the materials defined, all of which have a high surface area to total volume. Coating of the substrate is accomplished using any of the methods defined above with the matrix spread on a surface.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A liquid dispensing apparatus comprising: a container storing an aqueous sterile liquid, the container providing an interior volume; a dispensing means engaged with the container for expressing the sterile liquid from the container; a substrate of a porous, three-dimensional matrix immersed within the liquid, the substrate providing an anti-microbial coating providing at least 4 square centimeters of surface area for each cubic centimeter of the interior volume of the container.

2. The apparatus of claim 1 wherein the matrix is at least one of: small particles, and a wool-like material, the matrix compressed within a porous pocket.

3. The apparatus of claim 1 wherein the substrate is a high porosity sponge-like material.

4. The apparatus of claim 1 wherein the matrix is a high porosity tightly wound sheet stock.

5. The apparatus of claim 1 wherein the coating on the matrix is an antimicrobial agent or combination of agents.

6. The apparatus of claim 1 wherein the dispensing means is at least one of: a needle and syringe, an ophthalmic tip and a nozzle.

7. The apparatus of claim 1 wherein the coating is applied by at least one of: dip coating, spray coating, electroplating, plasma deposition, plasma spraying, and vacuum deposition.

8. The apparatus of claim 7 wherein the coating is held on the substrate by adsorption, absorption, diffusion, mechanical bonding, chemical bonding, co-polymerization, and blending.

9. A method for dispensing a liquid, the method comprising the steps of: providing a container storing a sterile aqueous liquid, the container providing an interior volume; engaging a dispensing means with the container for expressing the sterile liquid from the container; coating a matrix material with an antimicrobial coating with the matrix material disbursed or spread out, the coating providing at least 4 square centimeters of surface area for each cubic centimeter of the interior volume of the container; and compacting the matrix material into a porous, three-dimensional matrix immersed within the liquid.

10. The method of claim 9 further comprising the step of forming the matrix of at least one of: small spheres and a wool-like material, the matrix compressed within a porous pocket.

11. The method of claim 9 further comprising the step of forming the substrate from a high porosity sponge-like material.

12. The method of claim 9 further comprising the step of forming the matrix of a compressed high porosity sheet stock.

13. The method of claim 9 further comprising the step of forming the coating on the matrix of at least one of: an alcohol, an anilide, a metal salt, propiolactones, quanternary ammonium compounds, a biguanide, an iso-thiazolone, and a triclosan.

14. The method of claim 9 further comprising the step of forming the dispensing means of at least one of: a needle and syringe, an ophthalmic tip and a nozzle.

15. The method of claim 9 further comprising the step of coating the matrix with an antimicrobial agent or combination of agents.

16. The method of claim 15 further comprising the step of adhering the coating to the substrate by adsorption, absorption, diffusion, mechanical bonding, chemical bonding, co-polymerization, and blending.
